(19) United States
(12) Patent Application Publication
(10) Pub. No.: US 2016/0220724 A1
(43) Pub. Date: Aug. 4, 2016

(54) ABSORBENT POLYMER AND METHOD OF PREPARING THE SAME

(71) Applicants: SK GLOBAL CHEMICAL CO., LTD., Seoul (KR); SK INNOVATION CO., LTD, Seoul (KR)

(72) Inventors: Du-Youn KA, Daejeon (KR); Ju-Hee KIM, Daejeon (KR); Byoung-Tak YIM, Daejeon (KR); Ju-Eun JUNG, Daejeon (KR); Byoung-Cheon JO, Seoul (KR)

(21) Appl. No.: 15/011,731

(22) Filed: Feb. 1, 2016

(30) Foreign Application Priority Data
Jan. 30, 2015 (KR) 10-2015-0015494

(51) Int. Cl. A61L 15/60; A61L 15/28; A61L 15/26

(52) U.S. Cl. CPC A61L 15/60; A61L 15/26; A61L 15/28

(57) ABSTRACT

An absorbent polymer has an absorbency under pressure (AUL) ranging from 20 to 45 g/g; a phase angle (67 ) of swollen gel ranging from 3 to 30 degrees; and a decrease in phase angle ranging from 3 to 35%. Thereby, the absorbent polymer may have favorable gel elasticity under pressure after swelling, so as to reduce adhesion between swollen particles. Accordingly, even after absorbing the liquid to swell the absorbent polymer, the polymer may maintain excellent flow conductivity, thereby reducing a decrease in absorption ability of the absorbent polymer.

### CROSS-REFERENCE TO RELATED APPLICATION

This application claims priority to and the benefit of Korean Patent Application No. 10-2015-0015494, filed on Jan. 30, 2015, the disclosure of which is incorporated herein by reference in its entirety.

### BACKGROUND

1. Field of the Invention

The present invention relates to an absorbent polymer with favorable absorption property and a method of preparing the same.

2. Description of the Related Art

Super-absorbent polymer (‘SAP’) is an artificial polymer material capable of absorbing water of several tens to several hundreds of times the weight of the polymer. Also, the polymer material has high water-retention ability and can keep the absorbed water without releasing after absorbing the water, even though a pressure is somewhat applied thereto. Therefore, this polymer is broadly used in various products including hygienic products such as diapers, sanitary goods, etc.

With advanced performance of the hygienic products such as diapers as a major use of the super-absorbent polymer, excellent physical properties are required in various applications. More particularly, such physical properties may include free absorption, absorption rate, extractables, absorbency under pressure, water-retention ability, liquid permeability, or the like. In order to improve the above-described physical properties, a number of methods such as a process of increasing a cross-linkage density of the surface layer of the polymer, or the like, have been continuously proposed.

Among various physical properties, the extractables, liquid permeability, absorbency under pressure and/or water-retention ability associated with characteristics of the absorbent polymer swollen by urine or body fluid may induce unpleasant feelings in daily-life of a user wearing the product if these physical properties are inferior. The product including the absorbent polymer added thereto is exposed to urine or body fluid several times during use. In this regard, liquid penetration (‘flow conductivity’) of swollen gel under pressure is important. If the flow conductivity of the swollen absorbent polymer is low, only the part exposed to the urine or body fluid is locally swollen to increase a volume of some parts, or the urine or body fluid cannot be absorbed well in the polymer inside the product but flow over the surface layer of the polymer at the surface of the product, therefore, these problems may cause the user to have unpleasant feelings. However, with high flow conductivity, the liquid such as urine or body fluid may uniformly spread, and be absorbed throughout the absorbent polymer contained in the product, therefore, the user may feel more comfortable in using the product.

As such, the flow conductivity of the absorbent polymer is generally explained as a gel-blocking phenomenon under pressure. As the gel-blocking phenomenon under pressure is serious, the body fluid could not pass through swollen gel but be locally absorbed therein and, as a result, flow over the surface of the gel. This problem may become a very significant factor to deteriorate physical properties of the absorbent polymer.

U.S. Pat. No. 8,466,228 discloses a super-absorbent polymer composition having excellent liquid transfer ability in swollen state and high water-retention ability, which includes a polymer containing monoethylene unsaturated monomer partially polymerized therein. However, this patent has not proposed an alternative solution in regard to the above-described problems.

### SUMMARY

Accordingly, it is an object of the present invention to provide an absorbent polymer having excellent absorption property and a high flow conductivity achieved by improving elasticity of swollen absorbent polymer particles, so as to exhibit excellent physical properties even after swelling.

The above object of the present invention will be achieved by the following characteristics:

(1) A method of preparing an absorbent polymer, including: polymerizing a polymer composition, which includes acrylic monomer, polysaccharide and a cross-linking agent; and drying and grinding a hydrogel obtained by the above polymerization, wherein the polysaccharide is included in an amount of 0.1 to 20% by weight to the acrylic monomer in the polymer composition.

(2) The method according to the above (1), wherein the polysaccharide is at least one selected from a group consisting of alginate, kappa-carrageenan, iota-carrageenan, lambda-carrageenan, pectin, konjac (agar) and cellulose.

(3) The method according to the above (1), wherein the polysaccharide is included in an amount of 0.5 to 10% by weight to the acrylic monomer in the polymer composition.

(4) A method of preparing an absorbent polymer, including: polymerizing a polymer composition, which includes acrylic monomer and a cross-linking agent; mixing a hydrogel obtained by the polymerization with polysaccharide and kneading the same; and drying and grinding the kneaded hydrogel, wherein the polysaccharide is included in an amount of 0.1 to 20% by weight to the acrylic monomer in the polymer composition.

(5) The method according to the above (4), wherein the polysaccharide is at least one selected from a group consisting of alginate, kappa-carrageenan, iota-carrageenan, lambda-carrageenan, pectin, konjac (agar) and cellulose.

(6) The method according to the above (5), wherein the polysaccharide is included in an amount of 0.5 to 10% by weight to the acrylic monomer in the polymer composition.

(7) An absorbent polymer, having: an absorbency under pressure (AUL) ranging from 20 to 45 g/g; a phase angle (δ) of swollen gel ranging from 3 to 30 degrees; and a decrease in phase angle ranging from 3 to 35%.

(8) The absorbent polymer according to the above (7), wherein the absorbency under pressure (AUL) ranges from 30 to 45 g/g.

(9) The absorbent polymer according to the above (7), wherein the phase angle (δ) of swollen gel ranges from 3 to 20 degrees.

(10) The absorbent polymer according to the above (7), wherein the phase angle (δ) of swollen gel ranges from 3 to 10 degrees.

(11) The absorbent polymer according to the above (7), wherein the decrease in phase angle ranges from 5 to 35%.

(12) The absorbent polymer according to the above (7), wherein the decrease in phase angle ranges from 10 to 35%.

(13) The absorbent polymer according to the above (7), having a particle size distribution ranging from 100 to 1000 μm.

The absorbent polymer of the present invention may have favorable gel elasticity under pressure after swelling, so as to reduce adhesion between swollen particles. Accordingly, even after absorbing the liquid to swell the absorbent polymer, the polymer may maintain excellent flow conductivity, thereby reducing a decrease in absorption ability of the absorbent polymer.

### BRIEF DESCRIPTION OF THE DRAWINGS

The above and other objects, features and other advantages of the present invention will be more clearly understood from the following detailed description taken in conjunction with the accompanying drawing, in which:

FIG. 1 is a view schematically illustrating the configuration of an apparatus for measuring absorbency under pressure.

### DETAILED DESCRIPTION

The present invention discloses an absorbent polymer and a method of preparing the same. The absorbent polymer prepared according to the inventive method has: an absorbency under pressure (AUL) ranging from 20 to 45 g/g; a phase angle (δ) of swollen gel ranging from 3 to 30 degrees; and a decrease in phase angle ranging from 3 to 35%. Thereby, the absorbent polymer may have favorable gel elasticity under pressure after swelling, so as to reduce adhesion between swollen particles. Accordingly, even after absorbing the liquid to swell the absorbent polymer, the polymer may maintain excellent flow conductivity, thereby reducing a decrease in absorption ability of the absorbent polymer.

The absorbency under pressure (AUL) according to one embodiment of the present invention may refer to a value measured by Experimental Example 1 to be described below, wherein 6 g of sodium chloride (instead of saline), 4 g of potassium chloride, 0.6 g of calcium chloride, and 0.3 g of magnesium chloride were weighed, ultrapure water was added to make 1,000 g of solution, followed by stirring for 1 hour to form synthetic urine, and this urine was used.

According to one embodiment of the present invention, a phase angle (δ) of swollen gel means a relative ratio between viscosity modulus and elastic modulus, which was converted into an angle, wherein the above viscosity modulus and elasticity modulus were measured by introducing 1 g of the absorbent polymer into 20 g of the synthetic urine while rotating the same at 500 rpm, swelling the absorbent polymer 20 times (based on weight) to form a gel, then, using a parallel plate in an ARES rheometer under a condition of 5% strain at 35° C.

According to one embodiment of the present invention, a decrease in phase angle (%) means a relative increase in elasticity of the gel swollen by the synthetic urine to ultrapure water, which was obtained by swelling the absorbent polymer 20 times (based on weight) with ultrapure water and the synthetic urine, respectively, to prepare swollen gel, measuring a phase angle of the swollen gel, and substituting the measured value for Equation 1 below.

Decrease in phase angle (%)=[Phase angle(ultrapure water)−Phase angle(synthetic urine)]/Phase angle(ultrapure water)*100 [Equation 1]

According to one embodiment of the present invention, there is provided an absorbent polymer having the absorbency under pressure (AUL) in a range of 20 to 45 g/g, the phase angle of swollen gel (δ) in a range of 3 to 30 degrees, and the decrease in phase angle (%) in a range of 3 to 35%.

The absorbent polymer of one embodiment of the present invention satisfies a specified physical property, that is, the absorbency under pressure of 20 to 45 g/g when using the synthetic urine instead of saline.

When the absorbency under pressure of the absorbent polymer satisfies the above range, it is possible to contain a sufficient amount of water absorption enough to prevent a user of a product such as a diaper from having unpleasant feelings in case where the absorbent polymer is used for the above product. Further, there is particularly such an advantage that sufficient water-retention ability may be achieved to endure a pressure applied during daily-life activity.

If the absorbency under pressure of the absorbent polymer according to one embodiment of the present invention is less than 20 g/g, absorption property under pressure applied during daily-life activity, when a user wears an absorption product including the above absorbent polymer, is reduced to cause the user to have unpleasant feelings, or entails a problem of requiring the absorption product to be often changed. If the absorbency under pressure exceeds 45 g/g, moisture is excessively absorbed to decrease an intensity of the swollen gel, thus causing a problem that the gel is easily crushed and the absorbed water is eluted again. The absorbency under pressure of the absorbent polymer, for example, ranges from 30 to 45 g/g, in order to use the absorbent polymer as an absorbent for an absorption product.

In addition, the absorbent polymer according to one embodiment of the present invention may have a phase angle of swollen gel (δ) and a decrease in phase angle (%) in specified ranges thereof.

Gap blocking in the swollen gel may be explained by the following five (5) adhesion mechanisms between particles: 1) mechanical adhesion; 2) chemical adhesion; 3) adhesion by dispersion force; 4) electrical adhesion; and 5) adhesion by diffusion force.

The surface of the absorbent polymer has generally a negative charge to thus occur electrical repulsive force, therefore, the adhesion of the absorbent polymer cannot be explained by the electrical adhesion mechanism. In addition, since no chemical reaction occurs if the polymer is in swollen state, the chemical adhesion mechanism cannot also explain the above adhesion of the absorbent polymer. Accordingly, in order to suitably elucidate the adhesion of the swollen absorbent polymer, it is necessary to consider mechanical properties, dispersion force and/or diffusion force of the swollen polymer.

Among those, the mechanical mechanism may become a major cause of adhesion between particles in the absorbent polymer. Since the swollen absorbent polymer absorbs body fluid several tens of times the weight of the polymer, the absorbed water may occupy most of constitutional ingredients to thus considerably deteriorate mechanical properties. Accordingly, if a pressure is applied, a structure of the polymer is easily deformed, therefore, the particles are compactly adhered together under a pressure. Such mechanical deformation-based adhesion may be a major cause for gap blocking between swollen particles under pressure.

The absorbent polymer may have reduced flow conductivity since a gap between absorbent particles is blocked by adhesion thereof under a pressure condition after swelling. When the swollen gel receives a pressure, a shape of the gel is changed. If elasticity is low and viscosity property is high, the change in shape becomes serious and a gap between particles is decreased. If blocking occurs between particles, a liquid cannot flow inside but be locally absorbed or flow over the surface of the blocked swollen gel, therefore, a user wearing an absorption product containing the absorbent polymer may have unpleasant feelings. If the swollen gel does not have elastic property, the swollen gel may not return to its original condition even when the pressure is removed, and the particles are still adhered together and, thereafter, absorption property to liquid may be drastically deteriorated.

The present inventors have found that mechanical properties of the swollen gel are closely associated with flow conductivity, as described above. In particular, it could be found that a phase angle obtained from a ratio between a viscosity modulus and an elastic modulus of the swollen gel significantly relates to the flow conductivity of the swollen gel and, as a result, one embodiment of the present invention has been completely devised. More particularly, as the measured phase angle is reduced, the elasticity of the swollen gel may be increased. Further, it may be understood that, when the phase angle of the gel swollen by synthetic urine exhibits a higher decrease in phase angle, compared to the phase angle of the gel swollen with ultrapure water, the gel has excellent elastic property in the synthetic urine.

The absorbent polymer of one embodiment of the present invention may satisfy a specified physical property, that is, a phase angle of swollen gel in the synthetic urine in a range of 3 to 30 degrees (°).

If the phase angle of the swollen particle is less than 3 degrees, absorption ability is reduced. If the phase angle of the swollen particle exceeds 30 degrees, the gel is greatly deformed by a pressure applied thereto, hence causing adhesion of particles and blocking a gap between the particles. Accordingly, there is a problem of reducing the flow conductivity of the swollen absorbent polymer. In consideration of these aspects, the phase angle of the swollen particles may range from 3 to 20 degrees, and particularly, from 3 to 10 degrees.

Further, the absorbent polymer of one embodiment of the present invention may satisfy a specific physical property, that is, a decrease in phase angle of the gel swollen by the synthetic urine to ultrapure water in a range of 3 to 25%, according to the above Equation 1. If the decrease in phase angle of the absorbent polymer satisfies a value within the above range, the flow conductivity of the swollen gel may be greatly improved.

If the decrease in phase angle of the absorbent polymer according to one embodiment of the present invention is less than 3%, the gel swollen by the synthetic urine does not have sufficient elastic property, hence causing a problem of reducing flow conductivity. If the decrease in phase angle of the absorbent polymer exceeds 35%, elastic property is increased too much, hence causing a problem of reducing absorption ability. In consideration of these aspects, the decrease in phase angel may range from 5 to 35%, and particularly, from 10 to 35%.

According to another embodiment of the present invention, the absorbent polymer may satisfy a specific physical property, that is, a particle size distribution of 100 to 1000 μm.

When the particle size distribution of the absorbent polymer is within the above range, it is possible to prevent the particles from scattering easily due to so small size particles. In addition, even when an impact is applied during preparation of the absorbent polymer, the absorbent polymer is not easily crushed. Further, other problems in the manufacture of absorbent products having a uniform thickness may also be prevented.

Hereinafter, a method of preparing an absorbent polymer according to one embodiment of the present invention will be described in detail.

The method of preparing an absorbent polymer according to one embodiment of the present invention may include polymerization, drying and grinding processes. The inventive method may further include a surface cross-linking process and, optionally, a kneading process.

The polymerization process may be conducted by polymerizing a polymer composition including acrylic monomer and a cross-linking agent.

The polymer composition of one embodiment of the present invention may include acrylic monomer. Herein, the acrylic monomer refers to acrylic acid and salts thereof. The polymerization of acrylic acid may be performed by forming an acrylic salt through alkalization. For example, the treatment may be conducted using alkali-metal hydroxide, ammonia or organic amine. Among these, in order to prepare an absorbent polymer having excellent physical properties, acrylic acid is particularly treated with alkali-metal hydroxide, for example, sodium hydroxide, potassium hydroxide or lithium hydroxide. Also, in order to improve the absorption ability of the absorbent polymer, alkalization is particularly conducted such that a neutralization rate of acid groups in the acrylic acid reaches 60 mol. % or more.

The polymer composition according to one embodiment of the present invention may include a cross-linking agent. Such a cross-linking agent may include any one widely used in the related art and, in particular, may be selected among compounds having functional groups possibly reacting with a water-soluble substituent in a monomer. For example, the above cross-linking agent may be selected from a group consisting of bis-acrylamide having 6 to 12 carbon atoms, bis-methacrylamide, poly(meth)acrylate of polyol having 2 to 10 carbon atoms, and poly(meth)allylether of polyol having 2 to 10 carbon atoms, or the like, however, it is not particularly limited to the above listed compounds.

An amount of the cross-linking agent used herein is not particularly limited, but may range from 0.001 to 2 mol. %, and particularly, 0.005 to 0.5 mol. % to a total acrylic monomer included and polymerized in the polymer. If a content of the cross-linking agent is less than 0.001 mol. %, a cross-linkage density is too low to absorb moisture, instead, the cross-linking agent may be dissolved. If the content exceeds 2 mol. %, the cross-linkage density is too high, hence reducing expansion for water absorption and causing a difficulty in achieving desirable absorption effects.

The absorbent polymer provided by one embodiment of the present invention may satisfy physical properties described above by mixing the polymer composition with polysaccharide as an elasticity enhancer to improve elastic property of the swollen gel. Such polysaccharide may be prepared in a form of water-soluble solution and mixed with the polymer composition, and can be mixed in any of processes before or after polymerization, provided that the process proceeds before a drying process. For example, the elasticity enhancer may be mixed during polymerization or in a kneading process after polymerization.

The elasticity enhancer may be coagulated to form a primary rigid helix type structure, firstly, to exhibit excellent elasticity. Then, when the structure meets with cations including potassium and calcium, this structure may be coagulated again to form a secondary rigid aggregate as a cross-linkage point, secondly, to exhibit more excellent elastic property. More particularly, when the absorbent polymer containing the elasticity enhancer absorbs body fluid such as urine excreted from the body, the elasticity enhancer in the absorbent polymer meets with potassium or calcium cations contained in the body fluid, thus forming such aggregate as described above.

For evaluation of physical properties of the absorbent polymer, 0.9 NaCl saline prepared in consideration of only ion concentration is generally used. However, the above saline is different from the ion configuration of actual urine. Therefore, the present inventors have prepared synthetic urine by regulating concentrations of 0.06% magnesium chloride, 0.04% calcium chloride, 0.3% potassium chloride and 0.5% sodium chloride, which is substantially similar to the actual urine, then have utilized the prepared synthetic urine in order to concretely explain effects of the elasticity enhancer. The natural urine of a human being with the same constitutional compositions as the synthetic urine includes calcium or potassium ions to thus satisfy a condition for forming the secondary aggregate. However, if ultrapure water without potassium or calcium ions is absorbed, the elasticity enhancer does not form the secondary aggregate, therefore, improvement of elastic property may be expected by only the primary helix type structure formed of the elasticity enhancer. The absorbent polymer including polysaccharide as the elasticity enhancer may have excellent elastic property under pressure in swelling, compared to other polymer without the elasticity enhancer. Accordingly, the absorbent polymer is not easily deformed even when a pressure is applied to the absorbent polymer, therefore, a gap between particles is not blocked, while enables the body fluid to pass through the particles easily.

The polysaccharides described above are not particularly limited so long as these can be used for preparing an absorbent polymer, and types thereof are not particularly limited within a range not departing from the purpose of the present invention. In particular, polysaccharides used as a thickener for food may be exemplified. The thickener for food is used to provide a texture of food such as elasticity and has been fully proved to be safe. Accordingly, there is no problem in an aspect of safety even if the thickener for food is used in the production of an absorbent polymer. Such polysaccharides may include, for example, at least one of alginate, carrageenan, pectin, konjac (agar) and cellulose, however, they are not being particularly limited thereto. The carrageenan used herein may be kappa, lambda and iota carrageenan, which are used alone or in combination of two or more thereof.

The polysaccharides may be used in an amount of 0.1 to 20 by weight (‘wt. %’), and for example, 0.5 to 10 wt. % to the acrylic monomer in the polymer composition. Particularly, the polysaccharide is used in an amount of 1 to 5 wt. %. If the amount of polysaccharide is less than 0.1 wt. % to a total monomer in the polymer composition, the swollen gel has insignificant improvement of elasticity. If the amount of polysaccharide exceeds 20 wt. %, elasticity is too high to absorb moisture well, and a constitutional ratio of acrylic acid-based absorbent is decreased to reduce absorption ability of the absorbent polymer.

The polymer composition may have more appropriate physical properties for polymerization, when oxygen dissolved in a monomer ingredient under an inert gas atmosphere is substituted by such inert gas. This inert gas may be selected from, for example, nitrogen, carbon dioxide or argon gas.

Polymerization of the polymer composition may be performed by any one selected from thermal polymerization and photo-polymerization, or a combination of these two methods. More particularly, the thermal polymerization may be performed by selecting any one among typical heat polymerization to polymerize at a temperature of 40 to 90° C. for 2 to 30 minutes, or redox polymerization to polymerize at a relatively low temperature of 25 to 50° C. for 2 to 30 minutes. On the other hand, the photo-polymerization may be performed by irradiating UV-light at a temperature of 25 to 110° C. for 10 seconds to 20 minutes. When using the combination of the above both methods, a polymer composition including a photo-initiator and a thermal initiator mixed therein may undergo photo-polymerization by UV radiation to generate a neutralization heat, followed by thermal polymerization since the thermal initiator begins a reaction with the neutralization heat, thereby performing the polymerization. In order to produce a hydrogel phase polymer having low content of extractables and more excellent physical properties, the method using a combination of thermal initiator and photo-initiator is particularly selected.

The polymerization may be conducted by adding a polymerization initiator. The polymerization initiator added herein may be properly selected from conventional initiators used in the related art according to polymerization methods. The polymerization initiator used herein may include, for example, at least one initiator selected from a group consisting of azo-initiator, peroxide initiator, redox initiator, organic halide initiator, acetophenone, benzoin, benzophenone, benzyl compounds or derivatives thereof. A photo-polymerization initiator may include, for example, acetophenone, benzoin, benzophenone, benzyl compounds and derivatives thereof, in particular, at least one initiator selected from a group consisting of diethoxy acetophenone, 2-hydroxy-2-methyl-1-phenylpropan-on, 4-(2-hydroxyethoxy)phenyl-(2-hydroxy)-2-propylketone, 4-benzoyl-4′-methyl-diphenyl sulfide, azo-compounds, or the like.

An amount of the polymerization initiator used herein is not particularly limited but, for example, may range from 0.001 to 2 mol. %, and particularly, from 0.01 to 0.1 mol. % to a total monomer included and polymerized in the polymer. If the polymerization initiator is less than 0.001 mol. %, unreacted monomer residue may be increased. If the polymerization initiator exceeds 2 mol. %, polymerization may be difficult to control.

The hydrogel obtained by polymerizing the polymer composition may optionally undergo a kneading process.

According to another embodiment of the present invention, the hydrogel may be mixed with an elasticity enhancer during kneading. That is, when the polymer composition was polymerized without the elasticity enhancer, the elasticity enhancer may be mixed with the hydrogel during kneading. In this case, the kneading process may be conducted using a kneading device such as a kneader, mincer, planetary mixer and hammer mixer, etc. and, selection and use of the kneading device are not particularly limited so long as this device can uniformly mix the hydrogel and the elasticity enhancer. The elasticity enhancer added during kneading may be the same material as the elasticity enhancer added during the polymerization.

The hydrogel obtained after the kneading process may undergo a drying process to control a water-retention rate. During drying, a drying temperature and a drying time may be suitably selected under proper conditions on the basis of the water-retention rate of the prepared hydrogel. For example, the drying process proceeds under a temperature condition of 160 to 190° C. for 20 to 40 minutes. If the drying temperature is less than 160° C., dry effects are low to extend the drying time. If the drying temperature exceeds 190° C., the surface of the hydrogel is excessively dried and easily crushed to increase a content of fine powders. When the content of fine powders, a time of removing the fine powders may be extended to thus reduce productivity. The water-retention rate of the hydrogel obtained after the drying may range from 1 to 10% by weight.

The absorbent polymer may be generally ground and used in a form of powder. The dried hydrogel may be ground through a milling process, and such grinding may be conducted by any conventional milling method without particularly limitation in a technical configuration thereof so long as it may be used for grinding a resin. For example, the milling device such as a pin mill, hammer mill, screw mill, freezer miller, etc. may be used for grinding. In general, the absorbent polymer used for a product may have a particle size of 100 to 1,000 μm.

The ground absorbent polymer may further undergo treatment using a cross-linking agent after grinding, in order to adjust the surface cross-linkage density. Through such cross-linking, the absorbent polymer may have a higher particle intensity and improved absorbency under pressure.

The cross-linking agent is, for example, selected from a group consisting of diol or glycol compounds having 2 to 8 carbon atoms.

The diol compounds may include, for example, 1,3-propanediol, 2,3,4-trimethyl-1,3-pentanediol, 2-butene-1,4-diol, 1,4-butanediol, 1,3-butanediol, 1,5-pentanediol, 1,6-hexanediol, 1,2-cyclohexanedimethanol, polycarbonate polyol, etc., which may be used alone or in combination of two or more thereof.

The glycol compounds may include, for example, monoethylene glycol, diethyleneglycol, triethylene glycol, tetraethylene glycol, polyethyleneglycol, propylene glycol, dipropylene glycol, polypropylene glycol, glycerol, polyglycerol, etc., which may be use alone or in combination of two or more thereof.

Hereinafter, preferred embodiments are proposed to more concretely describe the present invention. However, the following examples are only given for illustrating the present invention and those skilled in the related art will obviously understand that various alterations and modifications are possible within the scope and spirit of the present invention. Such alterations and modifications are duly included in the appended claims.

### EXAMPLE 1

400 g of acrylic acid and 340 g of ultrapure water (Milli-Q integral 3; Millipore Co.) were mixed to prepare an acrylic acid mixture solution. After dissolving 70 mol. % of sodium hydroxide (NaOH) to acrylic acid in 400 g of ultrapure water and cooling the same to 10° C., the mixture was slowly introduced into the acrylic acid mixture solution. Nitrogen purging was conducted at 10° C. for 30 minutes, followed by adding 0.4915 g of potassium metabisulfite (K₂S₂O₈) and 0.2457 g of 1-hydroxycyclohexylphenylketone thereto. After adding 2.457 g of sodium hydrogen carbonate (NaHCO₃), UV light at 1 mw/cm² was rapidly irradiated for 1 minute. After removing the UV light, the mixture was left for 6 minutes to obtain a hydrogel. The obtained hydrogel was cut into pieces with a size of 1 cm³, carrageenan kappa was weighed to 0.1 wt. part to the acrylic acid, then, used to prepare a 10% solution. The prepared solution and the cut hydrogel were passed through a hood mixer (SFD(G); Shinsung Co.). The mixture obtained through the hood mixer was passed again through the hood mixer to completely knead the mixture. The resultant mixture was dried by a forced circulation drier (OF-02PW; Jeio Tech Co.). After increasing the temperature from an initial temperature of 30° C. up to 100° C. for 5 minutes and drying the same for 10 minutes, the temperature was again increased to 120° C. followed by drying for 10 minutes, increased 150° C. followed by drying for 10 minutes, and finally, increased 180° C. followed by drying for 25 minutes. In the chamber filled with the dried air, the sample was stored until the sample is cooled to room temperature. The cooled solid was ground and only particles having a size of 150 to 850 μm were selected using a mesh. Such grinding was conducted by a freezer/mill 6870 (SPEX SamplePrep Co.) under a liquid nitrogen atmosphere for 30 minutes. The selected particles were subjected to surface cross-linking using PCP-500 (Propylene carbonate polyol; SK Co.). After dissolving 4.23 g of surface cross-linking agent in 7 g of ethanol and gently adding 7 g of water thereto to prepare a surface cross-linking composition, the composition was uniformly mixed with the particles by a high-speed stirrer at a stirring intensity of “Low” (HMF-3260S; Hanil Co., Ltd.), followed by a reaction at 180° C. for 20 minutes, thereby preparing the absorbent polymer as a final product. Herein, the high-speed stirrer has a silicon blade rounded at its edge to prevent the absorbent polymer particles from being crushed by the blade.

### EXAMPLES 2 TO 7

The same procedures as described in Example 1 were conducted to prepare an absorbent polymer except that the elasticity enhancer is used with the content listed in Table 1 below.

### EXAMPLE 8

400 g of acrylic acid and 340 g of ultrapure water (Milli-Q integral 3; Millipore Co.) were mixed to prepare an acrylic acid mixture solution. After dissolving 70 mol. % of sodium hydroxide (NaOH) to acrylic acid and 0.1 wt. part of carrageenan (Aldrich Co.) to acrylic acid in 400 g of ultrapure water and cooling the same to 10° C., the mixture was slowly introduced into the acrylic acid mixture solution. Nitrogen purging was conducted at 10° C. for 30 minutes, followed by adding 0.4915 g of potassium metabisulfite (K₂S₂O₈) and 0.2457 g of 1-hydroxycyclohexylphenylketone thereto. After adding 2.457 g of sodium hydrogen carbonate (NaHCO₃), UV light at 1 mw/cm² was rapidly irradiated for 1 minute. After removing the UV light, the mixture was left for 6 minutes to obtain a hydrogel. The obtained hydrogel was cut into pieces with a size of 1 cm³, followed by drying in a forced circulation drier (OF-0.2PW; Jeio Tech Co.). After increasing the temperature from an initial temperature of 30° C. up to 100° C. for 5 minutes and drying the same for 10 minutes, the temperature was again increased to 120° C. followed by drying for 10 minutes, increased 150° C. followed by drying for 10 minutes, and finally, increased 180° C. followed by drying for 25 minutes. In the chamber filled with the dried air, the sample was stored until the sample is cooled to room temperature. The cooled solid was ground and only particles having a size of 150 to 850 μm were selected using a mesh. Such grinding was conducted by a freezer/mill 6870 (SPEX SamplePrep Co.) under a liquid nitrogen atmosphere for 30 minutes. The selected particles were subjected to surface cross-linking using PCP-500 (Propylene carbonate polyol; SK Co.). After dissolving 4.23 g of surface cross-linking agent in 7 g of ethanol and gently adding 7 g of water thereto to prepare a surface cross-linking composition, the composition was uniformly mixed with the particles by a high-speed stirrer at a stirring intensity of “Low” (HMF-3260S; Hanil Co., Ltd.), followed by a reaction at 180° C. for 20 minutes, thereby preparing the absorbent polymer as a final product. Herein, the high-speed stirrer has a silicon blade rounded at its edge to prevent the absorbent polymer particles from being crushed by the blade.

### EXAMPLES 9 TO 11

The same procedures as described in Example 8 were conducted to prepare an absorbent polymer except that the elasticity enhancer is used with the content listed in Table 1 below.

### EXAMPLES 12 AND 13

The same procedures as described in Example 1 were conducted to prepare an absorbent polymer except that alginate was used as the elasticity enhancer with the content listed in Table 1 below.

### EXAMPLES 14 AND 15

The same procedures as described in Example 8 were conducted to prepare an absorbent polymer except that alginate was used as the elasticity enhancer with the content listed in Table 1 below.

### COMPARATIVE EXAMPLE 1

The same procedures as described in Example 1 were conducted except that the elasticity enhancer was not added.

### COMPARATIVE EXAMPLES 2 AND 4

The same procedures as described in Example 1 were conducted to prepare an absorbent polymer except that types and amounts of the elasticity enhancer to be used are as described in Table 1 below.

### COMPARATIVE EXAMPLES 3 AND 5

The same procedures as described in Example 8 were conducted to prepare an absorbent polymer except that types and amounts of the elasticity enhancer to be used are as described in Table 8 below.

TABLE 1

| | Elasticity enhancer | | |
|---|---|---|---|
| | | Content (wt. % | |
| Section | Type | to total monomer) | Addition |
| Example 1 | Carrageenan kappa | 0.1 | Kneading |
| Example 2 | Carrageenan kappa | 0.5 | Kneading |
| Example 3 | Carrageenan kappa | 1 | Kneading |
| Example 4 | Carrageenan kappa | 5 | Kneading |
| Example 5 | Carrageenan kappa | 10 | Kneading |
| Example 6 | Carrageenan kappa | 15 | Kneading |
| Example 7 | Carrageenan kappa | 20 | Kneading |
| Example 8 | Carrageenan kappa | 0.1 | Polymerization |
| Example 9 | Carrageenan kappa | 1 | Polymerization |
| Example 10 | Carrageenan kappa | 5 | Polymerization |
| Example 11 | Carrageenan kappa | 20 | Polymerization |
| Example 12 | Alginate | 1 | Kneading |
| Example 13 | Alginate | 5 | Kneading |
| Example 14 | Alginate | 1 | Polymerization |
| Example 15 | Alginate | 5 | Polymerization |
| Comparative | — | — | — |
| Example 1 | | | |
| Comparative | Carrageenan kappa | 25 | Kneading |
| Example 2 | | | |
| Comparative | Carrageenan kappa | 25 | Polymerization |
| Example 3 | | | |
| Comparative | Alginate | 25 | Kneading |
| Example 4 | | | |
| Comparative | Alginate | 25 | Polymerization |
| Example 5 | | | |

### EXPERIMENTAL EXAMPLE

With regard to samples containing the same extractables, physical properties of the absorbent polymer prepared in each of the examples and comparative examples were measured by the following procedures, and measured results are shown in Table 2 below.

1. Determination of Absorbency Under Pressures (AUL)

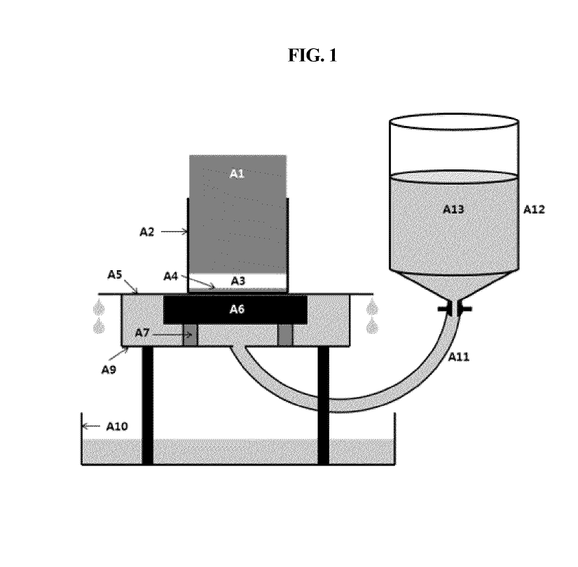

The absorbency under pressure was measured using the apparatus shown in FIG. 1

The measurement apparatus includes: A1: weight (0.3 psi), A2: cylinder, A4: non-woven fabric, A5: paper filter, A6: glass filter, A7: glass filter support, A9: cylinder support, A10: container, A11: connection line, A12: reservoir. Installation of the measurement apparatus and measurement of the absorbency under pressure were conducted as follows.

The cylinder support A9 and the reservoir A12 were connected by the connection line A11, and each of the devices had a hole through which 0.9% saline A13 contained in the reservoir can pass and move. After placing the cylinder support A9 on the container A10, the top of the glass filter A6 was matched with the top of the cylinder support A9 in the same height using the glass filter support A7. Thereafter, the paper filter A5 having a larger size than the top of the cylinder support A9 was positioned on the same. By opening a plug of the reservoir A12 to flow the saline A13, the saline A13 passing through the connection line was fully filled in the top of the cylinder support A9 and the excess of saline was naturally discarded into an external container through the paper filter A5. Air bubbles were removed if these bubbles are formed between the glass filter A6 and the paper filter A5.

After evenly spreading 0.9 g (w0) on a non-woven fabric A3 above the cylinder A2 covered with non-woven fabric A4 at the bottom thereof, the cylinder was placed on the paper filter and a weight A1 was quickly provided thereon.

After 1 hour, the hydrogel in the cylinder was recovered, followed by measuring the weight of the hydrogel (w1, weight of the absorbent polymer after absorption). From the measured weight, a weight of the measured sample (w0, weight of the absorbent polymer before absorption) was subtracted. The remainder was divided by the weight of the measured sample (w0) to calculate the absorbency under pressure.

Absorbency under pressure(g/g)=(Weight of absorbent polymer after absorption(w1)−Weight of absorbent polymer before absorption(w0))/Weight of absorbent polymer before absorption(w0). [Equation 2]

The saline used for measuring the absorbency under pressure was prepared as follows. After weighing 6 g of sodium chloride, 4 g of potassium chloride, 0.6 g of calcium chloride and 0.3 g of magnesium chloride, ultrapure was added to the above mixture to prepare a total weight of 1000 g, followed by agitating the same.

2. Measurement of Phase Angle

Using a parallel plate in an ARES rheometer (Advanced Rheometric Expansion System; TA Co.), a tan δ (at a shear rate of 100 rad/s) value of the obtained absorbent polymer was calculated in a dynamic frequency sweep mode, and the measured value was converted into an arctan value, that is, a phase angle (δ). The above measurement was performed after stacking the swollen gel on the parallel plate in 5 mm or more, and then, adjusting a gap of ARES to 1.8 mm to compactly stack the gel. A measurement temperature was 35° C. and the strain was 5%.

The swollen gel was obtained by quickly introducing 1 g of the prepared absorbent polymer between the center of vortex and a flask wall while rotating 20 g of the synthetic urine at 500 rpm, waiting until the absorbent polymer completely absorbs the synthetic urine, and then, swelling the same 20 times.

3. Determination of Decrease in Phase Angle (%)

Under the same measurement conditions as of the phase angle measurement, phase angles of the swollen gel in ultrapure water and the swollen gel in the synthetic urine, respectively, were measured. Then, these measured values were substituted for Equation 1 in order to calculate a decrease in phase angles.

4. Determination of Flow Conductivity

The flow conductivity of the obtained absorbent polymer was determined by the measurement method described in U.S. Pat. No. 8,466,228.

TABLE 2

| | Absor- | Phase | Phase | | |
|---|---|---|---|---|---|
| | bency | angle in | angle in | Decrease | Flow |
| | under | synthetic | ultrapure | in phase | conduc- |
| | pressure | urine | water | angle | tivity |
| Section | (g/g) | (degree) | (degree) | (%) | (*10⁻⁸ cm²) |
| Example 1 | 22.3 | 29.4 | 30.66 | 4.1 | 3.1 |
| Example 2 | 24.9 | 26.5 | 27.87 | 4.9 | 3.4 |
| Example 3 | 33.4 | 18.53 | 19.70 | 5.95 | 6.3 |
| Example 4 | 36.4 | 9.82 | 11.51 | 14.66 | 24 |
| Example 5 | 44.3 | 3.64 | 5.23 | 30.36 | 74.6 |
| Example 6 | 37.2 | 3.33 | 4.84 | 31.24 | 126.7 |
| Example 7 | 31.2 | 3.21 | 4.77 | 32.75 | 154.1 |
| Example 8 | 21.3 | 27.5 | 28.41 | 3.2 | 3.0 |
| Example 9 | 32.3 | 18.2 | 19.3 | 5.7 | 5.7 |
| Example 10 | 35.7 | 14.3 | 16.31 | 12.3 | 21 |
| Example 11 | 30.4 | 3.34 | 4.96 | 32.7 | 143 |
| Example 12 | 28.7 | 18.9 | 30.59 | 5.2 | 22.1 |
| Example 13 | 32.1 | 10.4 | 19.94 | 13.7 | 52.3 |
| Example 14 | 27.6 | 19.7 | 20.78 | 5.2 | 21.1 |
| Example 15 | 30.4 | 14.6 | 16.48 | 11.4 | 49.6 |
| Comparative | 16.3 | 34.23 | 34.83 | 0.71 | 0.7 |
| Example 1 | | | | | |
| Comparative | 15.8 | 1.37 | 2.26 | 39.37 | 230.4 |
| Example 2 | | | | | |
| Comparative | 14.4 | 1.88 | 3.06 | 38.5 | 211.4 |
| Example 3 | | | | | |
| Comparative | 15.7 | 2.21 | 3.63 | 39.2 | 214.1 |
| Example 4 | | | | | |
| Comparative | 14.2 | 2.01 | 3.25 | 38.1 | 205.7 |
| Example 5 | | | | | |

Referring to Table 2, it could be found that the absorbent polymer prepared in each of Examples 1 to 5 according to one embodiment of the present invention, which includes an elasticity enhancer in an amount of 0.1 to 20 wt. % to the acrylic monomer during the polymerization or kneading, exhibited a high flow conductivity of 3*10⁻⁸cm² or more while maintaining a high absorbency under pressure in a range of 20 to 45 g/g, compared to that of Comparative Example 1 without addition of an elasticity enhancer. If the flow conductivity is less than 3*10⁻⁸cm², liquid cannot be easily permeated but moisture is locally absorbed to increase a volume of some parts, or the liquid flows over the surface layer.

A decrease in phase angles of the swollen gels in the synthetic urine and ultrapure water, respectively, in each of Examples 1 to 15 according to one embodiment of the present invention was increased, compared to Comparative Example 1 without adding an elasticity enhancer. This means that the elasticity enhancer reacts with ions of the synthetic urine, thereby improving elasticity.

Accordingly, it is presumed that a product formed by applying the examples has excellent absorption ability and, even in swollen state, keep elasticity of the absorbent polymer to reduce adhesion between particles, thereby maintaining excellent absorption property.

Referring to Examples 1 to 7 of one embodiment of the present invention, it could be seen that, as a content of the elasticity enhancer added thereto is increased, the phase angle of swollen gel was reduced. This means an increase in elasticity of the swollen gel. Based on the measured flow conductivity, it could be seen that the elastic property was increased to improve flow conductivity. On the other hand, it could be found that the absorbency under pressure was increased up to a constant level when the content of the elasticity enhancer is increased to a specific value, and then, decreased inversely. The reason of this result may be considered because a constitutional ratio of the acrylic acid absorbent having high water-absorption ability becomes decreased to reduce absorption property.

The polymers in Comparative Examples 2 to 5, which include the elasticity enhancer of more than 20 wt. %, exhibited a very small phase angle. Even though these polymers have excellent flow conductivity, it could be found that a constitutional ratio of the acrylic acid absorbent is decreased due to excess of elasticity enhancer added thereto, resulting in reduction in absorption ability.

What is claimed is:

1. A method of preparing an absorbent polymer, the method comprising:
polymerizing a polymer composition, which includes acrylic monomer, polysaccharide and a cross-linking agent; and
drying and grinding a hydrogel obtained by the above polymerization, wherein the polysaccharide is included in an amount of 0.1 to 20% by weight to the acrylic monomer in the polymer composition.

2. The method according to claim 1, wherein the polysaccharide is at least one selected from a group consisting of alginate, kappa-carrageenan, iota-carrageenan, lambda-carrageenan, pectin, konjac (agar) and cellulose.

3. The method according to claim 1, wherein the polymer composition includes the polysaccharide in an amount of 0.5 to 10% by weight to the acrylic monomer.

4. A method of preparing an absorbent polymer, comprising:
polymerizing a polymer composition, which includes acrylic monomer and a cross-linking agent;
mixing a hydrogel obtained by the polymerization with polysaccharide and kneading the same; and
drying and grinding the kneaded hydrogel, wherein the polymer composition includes the polysaccharide in an amount of 0.1 to 20% by weight to the acrylic monomer.

5. The method according to claim 4, wherein the polysaccharide is at least one selected from a group consisting of alginate, kappa-carrageenan, iota-carrageenan, lambda-carrageenan, pectin, konjac (agar) and cellulose.

6. The method according to claim 5, wherein the polymer composition includes the polysaccharide in an amount of 0.5 to 10% by weight to the acrylic monomer.

7. An absorbent polymer, having: an absorbency under pressure (AUL) ranging from 20 to 45 g/g;
a phase angle (δ) of swollen gel ranging from 3 to 30 degrees; and
a decrease in phase angle ranging from 3 to 35%.

8. The absorbent polymer according to claim 7, wherein the absorbency under pressure (AUL) ranges from 30 to 45 g/g.

9. The absorbent polymer according to claim 7, wherein the phase angle (δ) of swollen gel ranges from 3 to 20 degrees.

10. The absorbent polymer according to claim 7, wherein the phase angle (δ) of swollen gel ranges from 3 to 10 degrees.

11. The absorbent polymer according to claim 7, wherein the decrease in phase angle ranges from 5 to 35%.

12. The absorbent polymer according to claim 7, wherein the decrease in phase angle ranges from 10 to 35%.

13. The absorbent polymer according to claim 7, having a particle size distribution ranging from 100 to 1000 μm.
